(19) United States
(12) Patent Application Publication
(10) Pub. No.: US 2020/0155632 A1
(43) Pub. Date: May 21, 2020

(54) COMPOSITION FOR TREATING AND PREVENTING MALE SEXUAL DISORDERS

(71) Applicant: KOLINPHARMA S.P.A., Milano (IT)

(72) Inventors: Emanuele LUSENTI, Milano (IT); Rita Paola PETRELLI, Milano (IT); Arianna DI LORENZO, Milano (IT); Valeria CURTI, Milano (IT); Francesco CAPPITELLI, Milano (IT)

(21) Appl. No.: 16/635,364

(22) PCT Filed: Aug. 2, 2018
(86) PCT No.: PCT/IB2018/055806

§ 371 Date: Jan. 30, 2020

(30) Foreign Application Priority Data
Aug. 2, 2017 (IT) 102017000089011

(51) Int. Cl. A61K 36/31; A61K 9/00; A61K 33/30; A61K 31/198; A61K 31/381; A61K 31/205; A61K 31/145; A61P 15/10; A23L 33/105; A23L 33/16; A23L 33/175

(52) U.S. Cl. CPC A61K 36/31; A61K 9/0056; A61K 33/30; A61K 31/198; A61K 31/381; A23V 2002/00; A61K 31/145; A61P 15/10; A23L 33/105; A23L 33/16; A23L 33/175; A61K 31/205

(57) ABSTRACT

A pharmaceutical composition or food supplement is disclosed that is effective in the treatment and the prevention of male sexual disorders such as erectile dysfunction and the decrease in sexual desire, comprising the synergic combination of zinc, citrulline, lipoic acid and/or N-acetyl-L-carnitine, and possible further active ingredients.

The invention relates to a pharmaceutical composition or food supplement for use in the treatment and the prevention of male sexual disorders, such as erectile dysfunction and/or the decrease in sexual desire.

Erectile dysfunction is defined as the persistent or recurrent inability to obtain or maintain an erection that is appropriate for completing the sexual act. Erectile dysfunction affects about 13% of the global population and 50% of men between 40 and 70 years of age. There are many causes of erectile dysfunction and they can be classified as psychogenic, organic and mixed. The most frequent psychogenic causes include performance anxiety, loss of sexual desire (decline of libido) and psychiatric disorders like depression and schizophrenia. The causes that are defined as organic, on the other hand, can be of various types:

endocrine, because they arise from hypogonadism and hyperprolactinaemia;

neurogenic, because they are consequences of neurological pathologies such as Alzheimer's disease, Parkinson's disease and multiple sclerosis;

iatrogenic, when they are caused by pharmacological treatment (psychotropic drugs, hormone treatments, antihypertensives);

vascular, because they are linked to cardiovascular pathologies such as hypertension, ischaemic stroke, and myocardial infarction.

Lastly, erectile dysfunction can occur in subjects affected by chronic pathologies such as diabetes and kidney failure. In fact, the population most at risk of erectile dysfunction includes subjects who are diabetic, obese, suffering from heart disease and subjects being treated with antidepressants.

Erection is a complicated process, which involves both the central nervous system and local messengers: in fact, the erection process comprises dilation of the penile arteries and mechanical compression of the emissary veins, with resulting accumulation of blood in the trabeculae and relaxation of the trabecular smooth muscle, mediated by the sympathetic nervous system.

From the molecular point of view, erection is supported by the release of nitric oxide (NO), which is produced in the sympathetic nerve ends by neuron nitric oxide synthase (nNOS) and in the vascular endothelium by endothelial NOS (eNOS). Relaxation of the trabecular smooth muscle is caused by a reduction in the concentration of intracellular calcium triggered by a second key messenger of the erection process, i.e. cyclic guanosine monophosphate (cGMP). cGMP synthesis is catalysed by the guanylate cyclase enzyme, which is activated by NO. The cGMP activates a cascade of intracellular events that culminate in a reduction of the concentration of cytoplasmic calcium ions, causing relaxation of the smooth muscle. At the vascular level, increased levels of cGMP result in vasodilation.

The levels of this second messenger are modulated by the phosphodiesterase enzymes, which break the phosphodiester bonds. This enzyme is present in 11 isoforms, which are present in different types of tissue. Isoform 5 (phosphodiesterase 5, PDE-5) is a key enzyme in the transduction of the signal NO/cGMP, because it catalyses the hydrolysis of the cGMP in its inactive metabolite 5′GMP. Today, the most favoured pharmacological treatment of erectile dysfunction uses drugs that inhibit PDE-5 (sildenafil, vardenafil, tadalafil) which, owing to the action mechanism thereof, are able to increase the intracellular levels of cGMP (1).

In certain cases, erectile dysfunction may be a symptom of another pathology that is more serious at the clinical level, above all cardiovascular pathologies. In fact, erectile dysfunction may indicate a more widespread disorder of the endothelium. Recently, some studies have shown that there is a connection between an improvement in erectile dysfunction and administering active ingredients that promote protection of the endothelium. One of the main factors causing damage to the endothelium is oxidative stress, which is defined as an imbalance between oxidant species (reactive oxygen species, ROS, and reactive nitrogen species, RNS) and endogenous antioxidant defences (both enzymatic and non-enzymatic). The accumulation of ROS in the corpus cavernosum and the penile endothelium determines the formation of the peroxynitrite radical from NO and superoxide anion, with two consequences: on the one hand, a major decrease of NO levels is detected, with a consequent reduction of the biological and functional properties thereof; on the other hand, the peroxynitrite radical reacts particularly aggressively with the endothelial cells, promoting oxidative damage to proteins, lipids and DNA, reduced NO synthesis, over-expression of proinflammatory cytokines and apoptosis of the endothelial cells, causing thinning of the endothelium and consequently reducing the quantity of bioavailable NO. On the basis of this reasoning, the use of antioxidant agents has been hypothesized in subjects suffering from erectile dysfunction to protect the endothelium from oxidative damage (2).

The object of the present invention is to provide a composition (pharmaceutical composition, food supplement or a composition for a medical device) that is effective in treating and preventing erectile dysfunction and other male sexual disorders, such as a decrease in sexual desire, and which consists of active ingredients of natural origin.

This object is achieved by the composition defined in appended claim 1.

The dependent claims define preferred features of the composition of the invention.

The appended claims are an integral part of the present description.

### DESCRIPTION OF THE FIGURES

FIG. 1 is a graph relating to the cell vitality test (MTT test) after 48 hours of treatment with the single active ingredients of the composition according to the invention in different concentrations.

FIG. 2 is a graph relating to the cell vitality test (MTT test) after 48 hours of treatment with the associations of active ingredients of a composition according to the invention (Menix composition).

FIG. 3 is a graph that illustrates the concentration levels (pM) of cGMP in ECV-304 cells following 48 hours of treatment with the single active ingredients of a composition according to the invention (Menix composition) and with the associations of said single active ingredients.

### DETAILED DESCRIPTION OF THE INVENTION

The composition of the invention is a pharmaceutical composition or a food supplement or medical device that is zinc-based, preferably in the form of zinc bisglycinate, L-citrulline, alpha lipoic acid and/or N-acetyl-L-carnitine, to which maca extract (Lepidium meyenii) and taurine can be added and which is particularly effective against disorders associated with erectile dysfunction, because the active ingredients contained therein have a synergic effect.

The object of the invention of the composition is thus a composition (pharmaceutical composition, food supplement or a composition for a medical device, known for short as a composition of the invention) comprising zinc, citrulline or a derivative or salt or precursor thereof and alpha lipoic acid or a salt thereof of acceptable pharmaceutical or food grade, and optionally excipients and/or binders and/or vehicles of an acceptable pharmaceutical or food grade.

Preferably, the composition of the invention comprises zinc, L-citrulline and alpha lipoic acid.

In one embodiment, the composition of the invention comprising as active ingredients zinc, citrulline, lipoic acid or a salt thereof of acceptable pharmaceutical or food grade and N-acetyl-L-carnitine or a different derivative (ester) of L-carnitine, preferably N-acetyl-L-carnitine.

Preferably, the composition of the invention comprises zinc, L-citrulline, alpha lipoic acid and N-acetyl-L-carnitine.

In one preferred embodiment, the composition of the invention comprising as active ingredients zinc, citrulline, alpha lipoic acid or a salt thereof of acceptable pharmaceutical or food grade, N-acetyl-L-carnitine or a different L-carnitine derivative, preferably N-acetyl-L-carnitine, taurine or a derivative or salt or precursor thereof and Andean maca extract (Lepidium meyenii Walp.), preferably dry aqueous Andean maca extract (Lepidium meyenii Walp.).

Preferably, the composition of the invention comprises zinc, L-citrulline, alpha lipoic acid, N-acetyl-L-carnitine, taurine and dry aqueous Andean maca extract (Lepidium meyenii Walp.).

Preferably, the composition of the invention comprises as active ingredients zinc bisglycinate, L-citrulline, alpha lipoic acid, N-acetyl-L-carnitine, taurine and dry aqueous Andean maca extract (Lepidium meyenii Walp.)

The composition of the present invention comprises citrulline or a derivative or salt or precursor thereof; preferably, the composition of the present invention comprises citrulline in the L-form. L-citrulline (IUPAC name: 2-amino-5-(carbamoylamino)pentanoic acid) is a non-essential amino acid (L-α-amino acid), which has proved to be particularly useful in treating erectile dysfunction, because it is involved in NO synthesis. L-citrulline is a precursor of L-arginine, a key amino acid in the production of NO because it is a substrate of the NOS enzyme. The secondary product of the reaction catalysed by the various NOS isoforms is L-citrulline, which at the renal level is converted into L-arginine to give rise to a new NO cycle. This recirculation has significant importance in maintaining NO production constant in the organism. Nevertheless, using L-citrulline as an NO donor is more advantageous than using L-arginine, as the latter is characterized by a shorter half-life and less bioavailability, because it is well metabolised to ornithine and urea by arginases in the intestinal lumen and in the liver, the concentration of which is greater in subjects affected by erectile dysfunction. L-citrulline, on the other hand, is not subject to presystemic elimination, but undergoes systemic metabolism. It is converted by argininosuccinate synthase to L-argininosuccinate and, subsequently, it is converted to L-arginine by argininosuccinate lyase. In the literature it has in fact been demonstrated that the oral administration of L-citrulline causes an increase in the blood levels of L-arginine compared with basic values (3). In addition, numerous studies conducted on both animal and human models have shown the efficacy of L-citrulline supplements in improving the erection process. Administering L-citrulline (2% p/v in water) for three weeks to rats with acute arteriogenic erectile dysfunction determined a significant improvement in erectile function, increasing NO plasmatic levels and preventing the damage caused by ischaemia to the muscle of the corpus cavernosum (4). The literature has provided clinical evidence of the efficacy of oral supplements of L-citrulline (1.5 g daily for a month) in 24 subjects suffering from mild erectile dysfunction, recording an increase in erectile function on the EHS (Erection Hardness Score) and IIEF-5 (International Index of Erectile Function) (5).

Lipoic acid was isolated for the first time in 1951 from liver extracts by the American biochemists L. J. Reed and I. C. Gunsalus. Lipoic acid is a molecule having a chiral centre and is produced by the human organism in the R chiral form (R-lipoic acid, IUPAC name (R)-5-(1,2-dithiolane-3-yl)pentanoic acid). Synthetic lipoic acid (also called alpha-lipoic acid) is on the other hand a mixture of the R and S chiral forms (racemic form). The composition of the present invention comprises lipoic acid in the racemic form thereof (alpha-lipoic acid and (RS)-lipoic acid) or, alternatively, in the R form thereof, (R-lipoic acid), preferably in the racemic form. Lipoic acid, which is also known as thioctic acid or vitamin N, is produced by our organism in small quantities, but can be absorbed, albeit in small quantities through the diet (broccoli, brewer's yeast and offal are the best sources). It is a molecule with a low molecular weight, which is characterized by good solubility in both hydrophilic and lipophilic environments, which is the reason why it can exert its biological and functional actions both in the aqueous (cytoplasmic) phase and in the lipid (cell membrane) phase of the cell. In nature, it exists in two forms: as cyclic disulphide (oxidized form) or as an open chain, known as dihydrolipoic acid, which shows two sulfhydryl groups; the two forms are easily interconvertible by oxidation-reduction reactions. Alpha-lipoic acid or R-lipoic acid is characterized by great antioxidant capacity that is due to its particular chemical structure and, mainly, to the presence of the disulfide bridge that acts as an electron acceptor. As an antioxidant, alpha-lipoic acid or R-lipoic acid not only acts as a scavenger of reactive oxygen species (ROS), but also has the chelating properties of transition metals. Recent studies have shown that NO-mediated vasodilation can be altered by high ROS levels that can react with NO, neutralizing the vasodilator activity (2) thereof. Using antioxidant molecules can accordingly improve the symptoms of erectile dysfunction by preventing both endothelial damage and the NO neutralization reaction. For this purpose, alpha-lipoic acid has been used in different studies in both an animal and human model. In the animal model of a diabetic rat, with a 41% reduction of NO-mediated vasodilation, treatment with alpha-lipoic acid (300 mg/kg/daily for a month) prevented and ameliorated corpus cavernosum dysfunctions (6). Subsequently, it was demonstrated that treatment with alpha-lipoic acid (65 mg/kg/daily for 15 days), in the model of a diabetic rat produced an increase in levels of endothelial NOS and of neuron NOS, enzyme isoforms that play a key role in the erection process (mediators of endothelial vasodilation and of stimulation of the nerve fibres) and an inducible reduction of the levels of NOS, helping in the treatment of erectile dysfunction. Furthermore, treatment with alpha-lipoic acid causes an improvement of the masson trichrome, an indicator of histological damage to the collagen fibres and smooth muscle (7). Lastly, administering 600 mg/daily of alpha-lipoic acid to 26 subjects suffering from erectile dysfunction (from mild to moderate) produced a tangible effect on erectile capacity because, in addition to improving certain biochemical parameters connected to the metabolic syndrome, a significant improvement was recorded for indicators connected to erectile dysfunction, which were measured by questionnaire IIEF-5, including: sexual desire, degree of satisfaction, orgasmic function, erectile function (8).

The composition of the present invention comprises L-carnitine in the form of N-acetyl-L-carnitine or in the form of a different derivative (preferably an ester) of L-carnitine; preferably, the composition of the present invention comprises N-acetyl-L-carnitine. N-acetyl-L-carnitine (NALC) ((R)-3-acetyloxy-4-trimethylammonio-butanoate) is an L-carnitine ester, a compound that is physiologically present in all mammals. The structure formula is illustrated below:

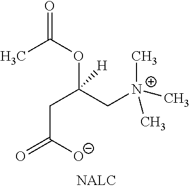

At the endogenous level, NALC is synthesized into L-carnitine owing to a transferase action. The main biological function of carnitine is to transport the long chain fatty acids from the cytoplasm to the mitochondrion, where they are subject to β-oxidation. This biological function is linked to the intracellular adjustment between acyl-CoA and acyl-carnitine, by the transfer of short-chain acyl groups from the inside of the mitochondrion to the cytoplasm. The availability of L-carnitine and of L-carnitine esters prevents the accumulation of fatty acids and acyl-CoA (respectively in the cytoplasm and in the mitochondrion) and, with the active transport of acetyl-CoA to the mitochondrion, stimulate the production of energy, promoting mitochondrial metabolism (9). Carnitines are able to exert this action also at the level of the sperm mitochondrion, because they accumulate in the epididymis in both free form and in acetylated form. Increasing the entry of fatty acids into the mitochondrion of the spermatozoon, the carnitines restore the phospholipid composition of the mitochondrial membranes, protect the DNA and the sperm membranes from possible oxidative damage. The proponionic derivative of the carnitine was used as an adjuvant (2 g daily) to treatment with sildenafil (twice weekly) because the contribution of the proponionic derivative of the carnitine at the endothelial level can reduce the percentage of non-responders to treatment with PDE-5 inhibitors. In fact, inducing the recovery of the vascular mechanical function to improve mitochondrial function, and administering propionyl-L-carnitine produced a significant reduction in the endogenous levels of endothelial dysfunction markers such as ICAM-1 and P-selectine (10).

It was also demonstrated that a NALC equivalent was able to act as a free radicals scavenger, thus reducing oxidative stress at the endothelial level, and limiting lipid peroxidation, performing a protective action at the vascular level. Protection of the endothelium thus results in an increase in the quantity of bioavailable NO and promotes the vasodilation process.

Zinc is an essential oligoelement, involved in more than 300 physiological functions, including immune defences, correct operation of sight, smell and memory, DNA transcription, protein translocation, and cell proliferation and differentiation. Zinc also plays a key role in maintaining normal levels of testosterone in the blood and maintaining good sperm quality: in fact, one of the main clinical signs of zinc deficiency are sexual dysfunctions such as reduced fertility and reduced sexual maturity (11). The composition of the invention comprises zinc, preferably in the form of zinc bisglycinate (chelated zinc bisglycinate) or in any other form that makes the zinc an element easily absorbable by the organism, in particular when the composition of the invention is administered orally.

Lepidium meyenii Walp. (maca root or Andean maca) is a root originating in the Andes belonging to the family of Brassicaceae, which are extremely rich in amino acids, magnesium, iodine and iron. In traditional Andean medicine, the root of Lepidium meyenii is used for its aphrodisiac properties and for its presumed ability to increase fertility. Pre-clinical studies support the use of Lepidium meyenii to improve sexual performance: in fact, administering a lipid extract of maca in the following dosages 45 mg/kg-180 mg/kg-1800 mg/kg improved erectile function in rats with erectile dysfunction (12). On the other hand, supplementing the normal murine diet with maca (2% of weight) increased the blood levels of testosterone and the steroidogenic ability of the Leydig cells with respect to rats fed a standard diet without maca (13). Scientific studies of an admittedly limited number of humans also converge in finding that the Andean root has the potential to exert a positive effect on subjects suffering from sexual dysfunctions. A double-blind randomized study lasting three months has shown that an extract of Lepidium meyenii is able to increase sexual desire (libido), without altering the plasma levels of testosterone and estradiol in men affected by moderate erectile dysfunction (14). A second randomized double blind clinical study characterized by a greater sample has shown that the administration of a dry extract of maca (2400 mg) for 12 weeks leads to a significant increase in the subjective perception of sexual well-being, increasing the IIEF-5 and the SAT-P (Satisfaction Profile) score linked to psychological, physical and social performances with respect to time zero (15). The composition of the invention can comprise an extract of Lepidium meyenii, preferably an aqueous dry extract obtained by standard extraction methods known to those skilled in the art, in particular standard water-based extraction methods.

The composition of the present invention comprises taurine or a derivative or salt or precursor thereof; the composition of the present invention preferably comprises taurine. Taurine, 2-aminoethanesulphonic acid, is one of the most abundant essential amino acids in the human body. Taurine is involved in numerous physiological functions, including detoxifying and antioxidant action, neuron development and modulation of neurotransmission, formation of bile, osmoregulation, regulation of calcium flows. Owing to the antioxidant properties of taurine, it can be used as an adjuvant in the treatment of sexual disorders. One experiment conducted on “aged” rats has shown that the administration of taurine (dissolved 1% in water) causes numerous improvements to testicular function because it increases the activity of enzymes that are markers of testicular function (sorbitol dehydrogenase, SDH, and glucose-6-phosphate dehydrogenase, G6PDH), increases the secretion of androgens and in particular of testosterone, mitigates local oxidative stress by reducing oxidative damage markers and increasing levels of endogenous antioxidants (superoxide dismutase, reduced glutathione, glutathione peroxidase). Furthermore, treatment with taurine increases levels of both NOS and NO in the rat's penis. Lastly, treatment with taurine improves sperm quality in terms of count, vitality and motility. Therefore, taurine is able to provide a protective action at the testicular level, mitigating oxidative stress and improving testicular circulation by affecting NO levels (16, 17).

The action of taurine on the eNOS/cGMP pathway has been partially demonstrated in the literature. Administering taurine (400 mg/kg/daily) to rats affected by type-1 diabetes increased the levels of both cGMP and eNOS at the level of the corpus cavernosum, leading to improved endothelial function. Increasing erectile function is also demonstrated by the increase in pressure inside the corpus cavernosum recorded after the administration of taurine (18).

The composition of the present invention can be, by way of non-limiting example, in liquid form, such as a solution, two-phase liquid system, suspension, syrup, semisolid form such as a gel, cream or foam or solid form such as powder, granules, flakes, aggregates, capsules, tablets, bars and equivalent forms.

The compositions of the invention are preferably formulated in a form that is suitable for oral administration, for example like hard or soft gelatin capsules, tablets, dissolving or chewable tablets, granules or powder in sachets, controlled release solid forms, chewing gum and the like.

According to particularly preferred embodiments, citrulline is present in the composition of the invention in a quantity comprised between 1 and 6000 mg of the total weight of the form of oral dosage, preferably comprised between 1000 mg and 5000 mg, more preferably comprised between 2000 mg and 4000 mg; zinc is present in the composition of the invention in a quantity comprised between 0.01 and 15 mg of the total weight of the form of oral dosage preferably comprised between 1 mg and 15 mg, more preferably comprised between 5 mg and 15 mg; alpha-lipoic acid is present in the composition of the invention in a quantity comprised between 1 and 2000 mg of the total weight of the form of oral dosage, preferably comprised between 50 mg and 1000 mg, more preferably comprised between 100 mg and 500 mg; N-acetyl-L-carnitine is present in the composition of the invention in a quantity comprised between 1 and 2000 mg of the total weight of the form of oral dosage, preferably comprised between 50 mg and 1000 mg, more preferably comprised between 200 mg and 600 mg; taurine is present in the composition of the invention in a quantity comprised between 1 and 1000 mg of the total weight of the form of oral dosage, preferably comprised between 50 mg and 1000 mg, more preferably comprised between 300 mg and 700 mg; and/or maca (Lepidium meyenii) is present in the composition of the invention in a quantity comprised between 1 and 3000 mg of the total weight of the form of oral dosage, preferably comprised between 50 mg and 1000 mg, more preferably comprised between 100 mg and 400 mg.

The compositions of the present invention are prepared according to conventional methods that are well known in the pharmaceutical field, using if necessary excipients, diluents, filling agents, and anti-caking agents selected on the basis of the desired form of dosage. Said excipients, diluents, filling agents and anti-caking agents are of acceptable pharmaceutical or food grade.

The present invention further provides a method of treatment of erectile dysfunction and/or the decrease in sexual desire in males and/or male sexual disorders in a subject in a state of need, in which said method of treatment involves administering to said subject an effective amount of the composition of the invention according to any one of the embodiments disclosed above, said administration is preferably oral and the composition of the invention is in one of the forms of administration indicated above.

Another object of the present invention is non-therapeutic use of the composition of the invention according to any one of the embodiments disclosed above for non-therapeutic treatment of erectile dysfunction and/or the decrease in sexual desire in males and/or male sexual disorders in a subject requiring such non-therapeutic treatment.

For the sake of clarity, in order to achieve the object of the present invention, the active ingredients of the composition of the present invention can be also administered separately (preferably in an interval of time from 30 minutes to 60 minutes) and in any order but, preferably, said active ingredients are administered to a subject simultaneously, and even more preferably in a single composition so as to obtain a more rapid effect and facilitate administration. When said active ingredients are administered in a single composition, this single composition corresponds to the composition of the present invention.

The following examples have been provided merely by way of non-limiting example of the scope of the invention as defined in the appended claims.

### EXAMPLES

The preferred form of dosage of the composition of the invention are granules for oral suspension (sachet). Nevertheless, the composition of the invention can be prepared in any form of dosage that is suitable for oral administration (for example, tablets, capsules, powder, solutions and oral suspensions).

### Formulation Example

The following table shows the composition (composition according to the invention) in active ingredients of a sachet weighing 6.5 g in total, including excipients.

| Component | mg/sachet |
|---|---|
| Citrulline | 3000 |
| N-acetyl-L-carnitine | 400 |
| L-taurine | 500 |
| Lipoic acid | 250 or 300 |
| Aqueous dry extract of Andean maca (Lepidium | 250 |
| meyenii Walp., maltodextrins, tuber) | |
| Zinc (from chelated zinc bisglycinate) | 12.5 (125% of NRV) |

The recommended dosage is one sachet a day for at least 60 days.

Study of activity: in vitro evaluation of the levels of cGMP expression

Introduction

The Menix product (as defined below), which falls within the scope of the present invention, is a food supplement based on L-citrulline, alpha-lipoic acid, zinc, taurine, N-acetyl-L-carnitine and Lepidium meyenii (mace), which is indicated for maintaining correct sexual and erectile function.

Erectile dysfunction is defined as the persistent or recurrent inability to obtain or maintain an erection that is appropriate for completing the sexual act. From a molecular point of view, erection is supported by the release of nitric oxide (NO), which is produced in the sympathetic nerve ends by neuron nitric oxide synthase (nNOS) and in the vascular endothelium by endothelial NOS (eNOS) Relaxation of the trabecular smooth muscle is caused by a reduction in the concentration of intracellular calcium triggered by a second key messenger of the erection process, i.e. cyclic guanosine monophosphate (cGMP). cGMP synthesis is catalysed by the guanylate cyclase enzyme, which is activated by NO. The cGMP activates a cascade of intracellular events that culminate in a reduction of the concentration of cytoplasmic calcium ions, causing relaxation of the smooth muscle. At the vascular level, increased levels of cGMP result in vasodilation [1]. Although erectile dysfunction can have different causes (psychogenic or organic), erectile dysfunction is nevertheless characterized by reduced levels of nitric oxide (NO), a powerful endogenous vasodilator, and cGMP. A second factor that plays an important role in the pathogenesis of erectile dysfunction is oxidative stress: at the endothelial level, accumulation of oxidant agents promotes apoptosis of the endothelium, causing a reduction in the local bioavailability of NO [2].

The active ingredients present in Menix assist an increase in the production of NO and cGMP and are able to exert a protective action to protect the vascular endothelium from oxidative damage.

Purpose

The purpose of this study is to evaluate the levels of expression of the cGMP in vascular endothelial cells following treatment with the active ingredients of Menix, both singly and in association.

Materials and Methods

The active ingredients contained in the Menix food supplement (composition according to the invention comprising the active ingredients listed below and excipients) are set out in Table 1 below, with the relative quantities for the single sachet.

TABLE 1

| Active ingredient | mg/sachet |
|---|---|
| L-citrulline | 3000 |
| alpha-lipoic acid | 250 or 300 |
| N-acetyl-L-carnitine | 400 |
| Taurine | 500 |
| Lepidium meyenii | 250 |
| Zinc | 12.5 |

Each active ingredient was tested singly and in association with others to evaluate the synergic activity.

Solubility Tests

Before being subjected to the cell assays, the active ingredients of Menix have been subjected to solubility tests to define the best solubilization agent for each of the assays. As the active ingredients have to be tested in cell cultures, in order to ensure complete compatibility, the solvents have been tested in the following order: culture medium, water, dimethyl sulphoxide (DMSO), organic solvents. Table 2 below shows the solvents in which each active ingredient has been solubilized.

TABLE 2

| Component | Solvent |
|---|---|
| Lipoic | methanol |
| Acid | |
| Zinc bisglycinate | Water |
| L-citrulline | Culture medium |
| L-Taurine | Culture medium |
| N-acetyl-L-carnitine | Water |
| Lepidium meyenii | ** |

**Lepidium meyenii was found to be insoluble in different solvents. In order to include the active ingredient in the assays, the powder was subjected to extraction with methanol (1:50, % p/v) overnight, under constant magnetic stirring, at ambient temperature and light-shielded.

Cell Cultures and Cell Vitality Tests (MTT)

For this experiment, human vascular endothelium cells were selected (ECV-304). The ECV304 were sowed in a T-75 flask (20000 cells/cm²) and maintained at 37° C. in an atmosphere humidified with 5% CO₂ in 199 culture medium (Sigma Aldrich), supplemented with 10% foetal bovine serum (FBS), L-glutamine (0.584 mg/ml), sodium pyruvate (0.11 mg/ml), penicillin (100 UI/ml), streptomycin (100 μg/ml) and amphotericin B (2.5 μg/ml). In each well, the culture medium was changed every 2-3 days, until the cells reached sub-confluence. At this point, the cells were treated with trypsin and EDTA and were re-examined in appropriate culture flasks.

For the cell vitality tests, ECV-304 cells were sowed (2000 cells/cm²) in multiwell cells consisting of 96 wells (Corning Costar). The samples were tested in physiological concentrations, selected on the basis of the data provided by the literature (19-30). The concentration ranges for every single active ingredient are set out in Table 3, whilst all the concentration levels tested for each active ingredient are listed below:

Citrulline: 2000 μM-1000 μM-500 μM-100 μM-50 μM-10 μM

Zinc: 0.1 mg/ml-0.01 mg/ml-0.001 mg/ml

Maca: 200 μg/ml-20 μg/ml-2 μg/ml-0.2 μg/ml-0.02 μg/ml

Taurine: 1000 μM-500 μM-100 μM-50 μM-10 μM

N-acetyl-L-carnitine: 50 μg/ml-10 μg/ml-1 μg/ml-0.1 μg/ml

alpha-lipoic acid: 5 μg/ml-2.5 μg/ml-0.5 μg/ml-0.05 μg/ml.

The samples were left in contact with the cells for 48 hours. After 48 hours, the cells were washed with PBS and 100 μl of 0.5 mg/ml of 3-(4,5-dimethylthiazol-2-yl)-2,5-diphenyltetrazolium bromide solution (MTT, Sigma Aldrich) was added to each well. After 3 hours, the supernatant was removed and 100 μl of DMSO was added to each well. Optical density was measured with a FLUOstar Omega microplate reader at 570 nm and 670 nm. Cell vitality (%) was calculated as follows: 100×(ODs/ODc), where ODs (OD₅₇₀-OD₆₇₀) is the average value of the optical density of the sample and ODc (OD₅₇₀-OD₆₇₀) is the average value of the optical density of the untreated cells (control).

Evaluation of cGMP Levels

The cGMP levels were determined by ELISA assay, following the operating instructions given in the protocol supplied by the manufacturer (cGMP ELISA KIT—STA 505-5, Cloud Clone Corp), running the assay in the acetylated embodiment to increase the sensitivity thereof.

For the experiment, the ECV-304 cells were sowed in MW-6 and treated for 48 hours with the active ingredients of Menix and associations thereof. After the 48 hours had elapsed, the supernatants were collected and frozen at −80° C. until use. The untreated cells were used as a control.

Results and Discussion

Cell Vitality

Cell vitality was determined by the MTT test, which enables the enzyme and metabolic activity of the cells to be measured. It is a test that is conventionally used to evaluate cell vitality.

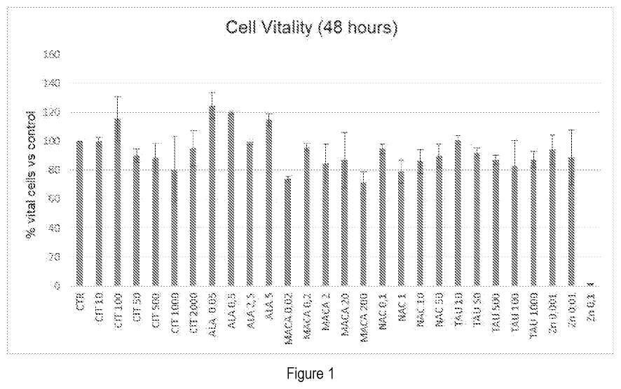

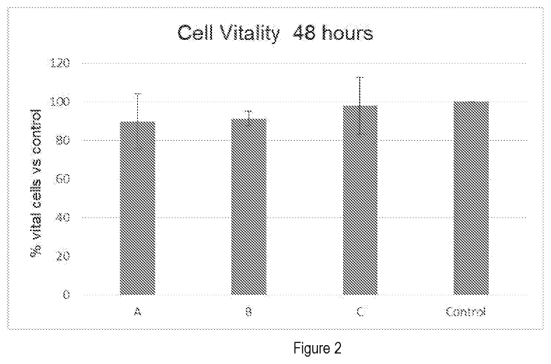

The active ingredients contained in Menix and the associations thereof were left in contact with the ECV304 cells for 48 hours, at the end of which cell vitality was evaluated. The results are set out in FIGS. 1 and 2. In particular, FIG. 1 is a graph illustrating the results of the cell vitality test (MTT test) after 48 hours of treatment with the single active ingredients in different concentrations. The results, expressed as the % of vital cells with respect to the control (non treated cells), are expressed as an average±standard deviation (n=3).

FIG. 2 is a graph illustrating the results of the cell vitality test (MTT test) after 48 hours of treatment with the associations of active ingredients of Menix with physiological concentrations. The results, expressed as the % of vital cells with respect to the control (non treated cells), are expressed as an average±standard deviation (n=3). A: zinc, citrulline, alpha-lipoic acid; B: zinc, citrulline, N-acetyl-L-carnitine; C: Menix food supplement.

Taking account of the results obtained by the cell vitality test and the optimum physiological concentrations, for the subsequent experiments, the concentrations indicated in Table 3 below were used for each active ingredient. In particular, Table 3 shows the tested concentration range for cell vitality for each active ingredient and the non cytotoxic physiological concentration used for the subsequent experiments.

TABLE 3

| | Concentration | | Concentration used | |
|---|---|---|---|---|
| Active ingredient | range for MTT | | in the experiment | |
| Citrulline (CIT) | 10-2000 | μM | 500 | μM |
| Zinc (Zn) | 0.1-0.001 | mg/ml | 0.01 | mg/ml |
| MACA | 0.02-200 | μg/ml | 0.2 | μg/ml |
| Taurine (Tau) | 10-1000 | μM | 100 | μM |
| N-acetyl-L-carnitine (NAC) | 0.1-50 | μg/ml | 10 | μg/ml |
| alpha-lipoic acid (ALA) | 0.05-5 | μg/ml | 0.5 | μg/ml |

Determination of the Levels of cGMP (Acetylated Version)

The base levels of cGMP were determined by ELISA assay, following instructions supplied by the manufacturer (STA 505-5 Cell Biolabs Inc).

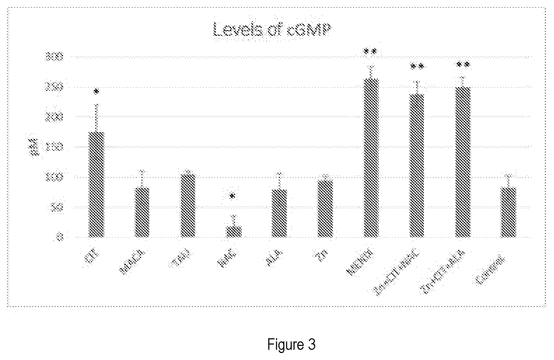

The results, expressed in terms of concentration (pM) of cGMP average±standard deviation (n=3 biological replicates), are set out in FIG. 3 and Table 4.

In particular, Table 4 below shows the levels of expression of cGMP (pM) in ECV-304 cells treated with the single active ingredients of Menix and associations thereof. The results are expressed as an average±standard deviation (n=3 biological replicates).

TABLE 4

| Samples | pM (cGMP) | SD |
|---|---|---|
| CONTROL | 82.1 | 0.5 |
| CITRULLINE | 140 | 1.5 |
| α-LIPOIC ACID | 60 | 16.2 |
| TAURINE | 137 | 5.48 |
| MACA | 80 | 18.4 |
| N-ACETYL-L- | 22 | 8.6 |
| CARNITINE | | |
| Zn | 90 | 9.9 |
| MENIX | 240 | 9.6 |
| Zn + Cit + ALA | 225 | 8.1 |
| Zn + Cit + NAC | 220 | 10.5 |

FIG. 3 is a graph that illustrates the concentration levels (pM) of cGMP in ECV-304 cells following 48 hours of treatment with the single active ingredients of Menix composition and associations thereof. The results are expressed as an average±standard deviation (n=3 biological replicates). *=p<0.05 vs control (Tukey post-hoc test); **=p<0.01 vs control (Tukey post-hoc test).

The statistical analysis (ANOVA) proved to be significant (F: 41.834; p <0.001). The results of the statistical comparison of the individual treatments with respect to the control, performed by the Tukey post-hoc test, showed the significances set out in Table 5 below.

TABLE 5

| Treatment | Tukey p value |
|---|---|
| Citrulline vs Control | p < 0.05 |
| NAC vs Control | p < 0.05 |
| Zn + Cit + ALA vs Control | p < 0.01 |
| Zn + Cit + NAC vs Control | p < 0.01 |
| Menix vs Control | p < 0.01 |

Determining Synergy

Synergy is defined as a combined action of two or more active ingredients that, in a mixture, exert a biological action that is greater than the sum of the actions of the single components of the mixture.

The synergic action of the active ingredients of Menix was evaluated on the basis of the percentage deviation of the average levels of cGMP from the control, which consists of non-treated cells.

Tables 6 and 7 below show the percentage deviation values from the control determined for the single active ingredients of Menix, for the sum of the single active ingredients and for the associations thereof.

In particular, Table 6 shows the percentage deviation values of the average levels of cGMP from the control (non-treated cells) following treatment with the active ingredients of Menix and associations thereof for 48 hours.

In particular, Table 7 shows the percentage deviation values of the levels of cGMP from the control (non-treated cells) arising from the sum of the individual active ingredients and associations thereof to evaluate the synergic effect.

TABLE 6

| | Average % deviation |
|---|---|
| | from |
| Samples | control |
| CITRULLINE | 70.52 |
| α-LIPOIC ACID | −26.92 |
| TAURINE | 66.87 |
| MACA | −2.56 |
| N-ACETYL-L-CARNITINE | −73.20 |
| Zn | 9.62 |
| MENIX | 192.33 |
| Zn + Cit + ALA | 174.06 |
| Zn + Cit + NAC | 167.97 |

TABLE 7

| | Average % deviation | Average % |
|---|---|---|
| | from sum of single | deviation |
| Combination | components | from mixture |
| MENIX | +44.33 | +192.33 |
| Zn + Cit + NAC | +6.94 | +167.97 |
| Zinc + Cit + ALA | +53.22 | +174.06 |

In all three cases, the action of the mixture is greater than the sum of the actions of the single components, thus indicating a synergic action in increasing cGMP production.

Conclusions

The active ingredients contained in Menix (L-citrulline, N-acetyl-L-carnitine, alpha-lipoic acid, taurine, maca and zinc) exert a synergic action on the increase in the levels of cGMP, which is an important mediator of the erection process.

The zinc-citrulline-alpha-lipoic acid and zinc-citrulline-N-acetyl-L-carnitine associations exert a synergic action on the increase of levels of cGMP, which is an important mediator of the erection process.

The three combinations indicated above further produce an increase in the levels of concentration of cGMP that is statistically significant with respect to the base levels. They can accordingly be considered to be valid agents for treating erectile dysfunction.

### REFERENCES

1) Clement P. Anatomy and physiology of genital organs—men. Handbook of Clinical Neurology, 2015, vol. 130, chapter 3.

2) Bielli A. Life Sciences, 2015, 143, 209-216.

3) Schwedhelm E. Br J Clin Pharmacol, 2007, 65:1, 51-59.

4) Shiota A. J Sex Med 2013, 10, 2423-2429.

5) Cormio L. UROLOGY 77 (1), 2011.

6) Keegan. Diabetologia 1999, 42, 343-350.

7) Hurdag C. Int J Tissue React 2005, XXVIII (3), 145-150.

8) Mitkov. Folia Medica 2013, 55(1), 55-63.

9) Calabrese. Journal of Nutritional Biochemistry 2006, 17, 73-88.

10) Morano. European Urology 2007, 52, 1768-1776.

11) Omu. Med Princ Pract 2008, 17, 108-116.

12) Zheng. Urology 2000, 55(4).

13) Ohta. Andrologia 2016, 48, 347-354.

14) Gonzales. Journal of Endocrinology, 2003, 176, 163-168.

15) Zenico. Andrologia 2009, 41, 95-99.

16) Yang. Amino Acids, 2015, 47, 1549-1558.

17) Yang J. Chapter 32 Taurine Enhances the Sexual Response and Mating Ability in Aged Male Rats. (A. El Idrissi and W. J. L'Amoreaux (eds.), Taurine 8, Advances in Experimental 347 Medicine and Biology 776, DOI 10.1007/978-1-4614-6093-0_32)

18) Ruan. J Sex Med, 2016, 1-8.

19) Morita M. et al, Immunology, Endocrine & Metabolic Agents in Medicinal Chemistry, 2013, 13, 214-220.

20) Suzuki T. et al, Journal of the International Society of Sports Nutrition, 2016, 13(6).

21) Breuillard C. et al., British Journal of Nutrition, 2015, 113, 120-124

22) Troncon Rosa F. et al, European Journal of Nutrition, 2014, 53, 823-830.

23) Van Stijn M. F. M. et al, International Journal of Molecular Sciences, 2015, 16, 12288-12306.

24) Sun Q. et al, Hypertension, 2016, 67, 541-549.

25) Baek S. M. et al, International journal of clinical pharmacology and therapeutics, 2015, 53(11), 980-986.

26) Kwon O. S. et al, Archives of Pharmacal Research, 2004, 27(6), 676-681.

27) Carlson D. A. et al, Alternative Medicine Review, 2007, 12(4), 343-351.

28) Hermann R. et al, Clinical Pharmacology: Advances and Applications, 2014, 6, 195-204.

29) Maret W. et al, Journal of Trace Elements in Medicine and Biology, 2006, 20, 3-18

30) FAO, World Health Organization, Human Vitamins and Mineral Requirements.

1. Pharmaceutical composition or food supplement or medical device comprising as active ingredients zinc, citrulline and lipoic acid or an acceptable pharmaceutical or food grade salt thereof, and optionally excipients and/or binders and/or vehicles of an acceptable pharmaceutical or food grade.

2. Pharmaceutical composition or food supplement according to claim 1, comprising as active ingredients zinc, citrulline, lipoic acid or an acceptable pharmaceutical or food grade salt thereof and N-acetyl-L-carnitine or a different L-carnitine derivative, preferably comprises zinc, L-citrulline, alpha lipoic acid and N-acetyl-L-carnitine.

3. Pharmaceutical composition or food supplement according to claim 1 or 2, comprising as active ingredients zinc, citrulline, lipoic acid or an acceptable pharmaceutical or food grade salt thereof, N-acetyl-L-carnitine or a different L-carnitine derivative, taurine and Andean maca extract (Lepidium meyenii Walp.); preferably comprises zinc, L-citrulline, alpha lipoic acid, N-acetyl-L-carnitine, taurine and Andean maca extract (Lepidium meyenii Walp.).

4. Pharmaceutical composition or food supplement according to any one of the preceding claims, wherein zinc is present in the composition in the form of bisglycinate zinc.

5. Pharmaceutical composition or food supplement according to any one of the preceding claims, comprising as active ingredients zinc bisglycinate, L-citrulline, alpha lipoic acid, N-acetyl-L-carnitine, taurine and Andean maca extract (Lepidium meyenii Walp.).

6. Pharmaceutical composition or food supplement according to any one of the preceding claims, wherein said pharmaceutical composition or food supplement is in a dosage form for oral administration.

7. Pharmaceutical composition or food supplement according to claim 6, wherein citrulline is present in a weight concentration comprised within a range from 1 mg to 6000 mg of the total weight of the oral dosage form, preferably comprised in the range from 1000 mg to 5000 mg, more preferably comprised in the range from 2000 mg to 4000 mg.

8. Pharmaceutical composition or food supplement for use according to claim 6 or 7, wherein the zinc is present in a weight concentration comprised within a range from 0.01 mg to 15 mg of the total weight of the oral dosage form, preferably comprised in the range from 1 mg to 15mg, more preferably comprised in the range from 5 mg to 15mg.

9. Pharmaceutical composition or food supplement for use according to any one of claims 6 to 8, wherein the alpha lipoic acid or an acceptable pharmaceutical or food grade salt thereof is present in a weight concentration comprised within a range from 1 mg to 2000 mg of the total weight of the oral dosage form, preferably comprised in the range from 50 mg to 1000 mg, more preferably comprised in the range from 100 mg to 500 mg.

10. Pharmaceutical composition or food supplement for use according to any one of claims 6 to 9, wherein N-acetyl-L-carnitine or a different L-carnitine derivative, preferably N-acetyl-L-carnitine, is present in a weight concentration comprised within the range 1-2000 mg of the total weight of the oral dosage form, preferably comprised in the range from 50 mg to 1000 mg, more preferably comprised in the range from 200 mg to 600 mg.

11. Pharmaceutical composition or food supplement for use according to any one of claims 6 to 10, wherein taurine is present in a weight concentration comprised within the range 1-1000 mg of the total weight of the oral dosage form, preferably comprised in the range from 50 mg to 1000 mg, more preferably comprised in the range from 300 mg to 700 mg.

12. Pharmaceutical composition or food supplement for use according to any one of claims 6 to 11, wherein the Andean maca extract (Lepidium meyenii Walp.) is present in a weight concentration comprised within the range 1-3000 mg of the total weight of the oral dosage form, preferably comprised in the range from 50 mg to 1000 mg, more preferably comprised in the range from 100 mg to 400 mg.

13. Pharmaceutical composition or food supplement according to any one of claims 1 to 12 for use in a method for the curative and/or preventive treatment of erectile dysfunction and/or the decrease in sexual desire in males.

14. Non therapeutic use of a composition according to any one of claims 1 to 12 for non-therapeutic treatment of erectile dysfunction and/or the decrease in sexual desire in males in a subject requiring such non-therapeutic treatment.
